(19) United States
(12) Patent Application Publication
(10) Pub. No.: US 2024/0010928 A1
(43) Pub. Date: Jan. 11, 2024

(54) USE OF BLEND OF WASTE PLASTIC WITH BIO FEED FOR CIRCULAR ECONOMY POLYPROPYLENE PRODUCTION

(71) Applicant: Chevron U.S.A. Inc., San Ramon, CA (US)

(72) Inventors: Joel E. SCHMIDT, San Ramon, CA (US); Tengfei LIU, San Ramon, CA (US); Hye-Kyung C. TIMKEN, Albany, CA (US)

(73) Assignee: Chevron U.S.A. Inc., San Ramon, CA (US)

(21) Appl. No.: 18/349,300

(22) Filed: Jul. 10, 2023

Related U.S. Application Data
(60) Provisional application No. 63/359,588, filed on Jul. 8, 2022.

(51) Int. Cl. C10G 11/05; C07C 4/06; C08J 11/10; B01D 3/14

(52) U.S. Cl. CPC C10G 11/05; C07C 4/06; C08J 11/10; B01D 3/14; C10G 2400/02; C10G 2400/04; C10G 2400/08; C10G 2300/1003; C10G 2300/1014; C10G 2300/1018; C07C 2529/12; C07C 2529/40; C08J 2323/12

(57) ABSTRACT

Provided is a continuous process for converting waste plastic into recycle for polypropylene polymerization. The process comprises selecting waste plastics containing polyethylene and/or polypropylene and preparing a blend of a bio feedstock and the selected plastic. The amount of plastic in the blend comprises no more than 20 wt. % of the blend. The blend is passed to a FCC unit. A liquid petroleum gas LPG olefin/paraffin mixture and naphtha are recovered from the FCC unit and can be passed on to make polypropylene.

### CROSS REFERENCE TO RELATED APPLICATIONS

The present application claims priority to U.S. Provisional Application No. 63/359,588, filed Jul. 8, 2022, the complete disclosure of which is incorporated herein by reference in its entirety.

### BACKGROUND

As part of efforts to reduce global warming, the industry is experiencing a rapid increase of chemicals and fuels production from renewable sources such as bio feedstocks or bio oils.

On the other hand, the world has seen extremely rapid growth of plastics production. According to Plastics Europe Market Research Group, the world plastics production was 335 million tons in 2016, 348 million tons in 2017, 359 million tons in 2018, and 367 million tons in 2020. According to McKinsey & Company, the global plastics-waste volume is estimated to be 460 million tons per year by 2030 if the current trajectory continues.

Single use plastic waste has become an increasingly important environmental issue. At the moment, there appear to be few options for recycling polyethylene and polypropylene waste plastics to value-added chemical and fuel products. Currently, only a small amount of polyethylene and polypropylene is recycled via chemical recycling, where recycled and cleaned polymer pellets are pyrolyzed in a pyrolysis unit to make fuels (naphtha, diesel), stream cracker feed or slack wax. The majority, greater than 80%, is incinerated, land filled or discarded.

The current method of chemical recycling via pyrolysis cannot make a big impact for the plastics industry. The current pyrolysis operation produces poor quality fuel components (naphtha and diesel range products), but the quantity is small enough that these products can be blended into fuel supplies. However, this simple blending cannot continue if very large volumes of waste polyethylene and polypropylene is to be recycled to address environmental issues. The products as produced from a pyrolysis unit are of too poor quality to be blended in large amounts in transportation fuels.

Processes are known which convert waste plastic into hydrocarbon lubricants. For example, U.S. Pat. No. 3,845,157 discloses cracking of waste or virgin polyolefins to form gaseous products such as ethylene/olefin copolymers which are further processed to produce synthetic hydrocarbon lubricants. U.S. Pat. No. 4,642,401 discloses the production of liquid hydrocarbons by heating pulverized polyolefin waste at temperatures of 150-500° C. and pressures of 20-300 bars. U.S. Pat. No. 5,849,964 discloses a process in which waste plastic materials are depolymerized into a volatile phase and a liquid phase. The volatile phase is separated into a gaseous phase and a condensate. The liquid phase, the condensate and the gaseous phase are refined into liquid fuel components using standard refining techniques. U.S. Pat. No. 6,143,940 discloses a procedure for converting waste plastics into heavy wax compositions. U.S. Pat. No. 6,150,577 discloses a process of converting waste plastics into lubricating oils. EP0620264 discloses a process for producing lubricating oils from waste or virgin polyolefins by thermally cracking the waste in a fluidized bed to form a waxy product, optionally using a hydrotreatment, then catalytically isomerizing and fractionating to recover a lubricating oil.

Other documents which relate to processes for converting waste plastic into lubricating oils include U.S. Pat. Nos. 6,288,296; 6,774,272; 6,822,126; 7,834,226; 8,088,961; 8,404,912 and 8,696,994; and U.S. Patent Application Publication Nos. 2021/0130699; 2019/0161683; 2016/0362609; and 2016/0264885. The foregoing patent documents are incorporated herein by reference in their entirety.

Globally, recycling or upcycling of plastic waste has gained great interest to save resources and the environment. Mechanical recycling of plastic waste is rather limited due to different types, properties, additives, and contaminants in the collected plastics. Usually, the recycled plastics are of degraded quality. Chemical recycling to the starting material or value-added chemicals has emerged as a more desirous route.

However, in order to achieve chemical recycling of single use plastics in an industrially significant quantity to reduce its environmental impact, more robust processes are needed. The improved processes should establish “circular economy” for the waste polyethylene and polypropylene plastics where the spent waste plastics are recycled effectively back as starting materials for the polymers or value-added chemicals or fuels. The establishment of such a circular economy while also employing renewable sources such as bio feedstocks would even further enhance the environmental benefits of such a recycling process.

### SUMMARY

Provided is an integrated process for converting plastic waste into recycle for polypropylene production. The process comprises selecting waste plastics to blend with a bio feedstock, with the waste plastics/bio feedstock blend then fed to and converted in a conversion unit. The conversion process produces clean monomers for propylene polymerization, as well as chemical intermediates. In one embodiment, the blend comprises about 20 wt % or less of the selected waste plastic. In another embodiment, the blend is fed to a refinery conversion unit, such as a FCC unit.

The term “bio” refers to biochemical and/or natural chemicals found in nature. Thus, a bio feedstock or bio oil would comprise such natural chemicals. The preferred starting bio feedstocks for the blend preparation include triglycerides and fatty acids, plant-derived oils such as palm oil, canola oil, corn oil, and soybean oil, as well as animal-derived fats and oils such as tallow, lard, schmaltz (e.g., chicken fat), and fish oil, and mixtures of these.

The incorporation of the process with an oil refinery is an important aspect of the present process and allows the creation of a circular economy with a single use waste plastic such as polypropylene. Thus, the blend is passed to a refinery FCC unit. The blend is passed at a temperature above its pour point in order to be able to pump the blend to the refinery FCC unit. The blend is heated above the melting point of the plastic before being injected into the reactor. A liquid petroleum gas C₃ olefin/paraffin mixture is recovered from the FCC unit. The C₃ olefin/paraffin mixture is separated into C₃ paraffin and C₃ olefin fractions. The C₃ olefin is passed to a propylene polymerization reactor to produce polypropylene. Optionally the C₃ paraffin is sent to a dehydrogenation unit to produce additional propylene, which can be used to produce polypropylene.

The refinery will generally have its own hydrocarbon feed flowing through the refinery units. An important aspect of the present process is not to negatively impact the operation of the refinery. The refinery must still produce valued chemicals and fuels. Otherwise, the incorporation of the process with an oil refinery would not be a workable solution. The flow volume must therefore be carefully observed.

The flow volume of the waste plastic/bio feedstock blend to the refinery units can comprise any practical or accommodating volume % of the total flow to the refinery units. Generally, the flow of the blend can be up to about 100 vol. % of the total flow, i.e., the blend flow is the entire flow, with no refinery flow. In one embodiment, the flow of the blend is an amount up to about 50 vol. % of the total flow, i.e., the refinery flow and the blend flow.

Among other factors, it has been found that a blend of waste plastic and a bio feedstock can be prepared, which blend can be made sufficiently stable to be stored or transported if desired. Further, the blend can then be converted in a conversion unit to value-added chemicals and fuels. The use of a bio feedstock together with waste plastic greatly enhances the environmental aspects of the conversion and recycling process. By further having the conversion unit part of a refinery operation, one can efficiently and effectively recycle plastic waste while also complementing the operation of a refinery in the preparation of higher value products such as gasoline, jet fuel, base oil and diesel. But also, by adding refinery operations it has been found that clean LPG (propane, propylene, butanes, and butenes) and naphtha can be efficiently and effectively produced from the waste plastics for ultimate polypropylene polymer production. Positive economics are realized for the overall process from recycled plastics to a polymer product with product quality identical to that of virgin polymer, while also enhancing the environmental aspects of the recycle process by employing a blend of a bio feedstock and waste plastic.

### BRIEF DESCRIPTION OF THE DRAWINGS

FIG. 1 depicts the current practice of pyrolyzing waste plastics to produce fuel or wax (base case).

FIG. 2 depicts a present process of preparing a hot homogeneous liquid blend of plastic and bio feedstock, and the feeding of the blend to a conversion unit.

FIG. 3 depicts in detail a stable blend preparation unit process, and how the stable blend can be fed to a conversion unit.

FIG. 4 depicts the plastic type classification for waste plastics recycling.

FIG. 5 depicts a present process where the prepared blend is passed to a conversion unit in a refinery to create value added chemicals and fuels, as well as chemicals for preparing recycled polypropylene.

FIG. 6 depicts a present process for establishing a circular economy for waste plastics where the plastic/bio oil blend is passed to a refinery FCC feed pretreater and then a refinery FCC unit.

FIG. 7 graphically depicts a thermal gravimetric analysis (TGA) of the thermal stability of polyethylene and polypropylene.

### DETAILED DESCRIPTION

In the present process, provided is a method for recycling waste plastics such as polyethylene and/or polypropylene back to value added chemicals and fuels, as well as virgin polypropylene. A substantial portion of polyethylene and polypropylene polymers are used in single use plastics and get discarded after its use. The single use plastic waste has become an increasingly important environmental issue. At the moment, there appear to be few options for recycling polyethylene and polypropylene waste plastics to value-added chemicals and fuel products. Currently, only a small amount of polyethylene/polypropylene is recycled via chemical recycling, where recycled and cleaned polymer pellets are pyrolyzed in a pyrolysis unit to make fuels (naphtha, diesel), steam cracker feed or slack wax.

Polypropylene is used widely in various consumer and industrial products. Polypropylene is the second-most widely produced commodity plastic after polyethylene with its mechanical ruggedness and high chemical resistance. Polypropylene is widely used in packaging, film, fibers for carpets and clothing, molded articles and extruded pipes. Today, only a small portion of spent polypropylene products is collected for recycling, due to the inefficiencies and ineffectiveness of the recycling efforts discussed above.

A process for recycling plastic waste back to clean monomer is now provided wherein waste plastic and a bio feedstock are simultaneously converted in a conversion unit. The clean monomers can be used for value added chemicals, fuels, as monomers for polymerization, e.g., recycling polypropylene. The process comprises preparing a novel blend of waste plastic and a bio feedstock. The blend is converted in a conversion unit, such as a catalytic process unit.

The integrated processes produce feedstocks to make clean monomer for polymerization. The integrated processes generate clean, recycled, propane, and propylene. The clean, recycled propane and propylene streams can be used for polypropylene production. The quality of the final polypropylene product is not diminished due to recycling of plastic waste.

In parallel, high-quality gasoline, jet and diesel fuel can be produced in a refinery from the waste plastics. The fuel components are upgraded in appropriate refinery units via chemical conversion processes. The final transportation fuels produced by the integrated process are high quality and meet the fuels quality requirements.

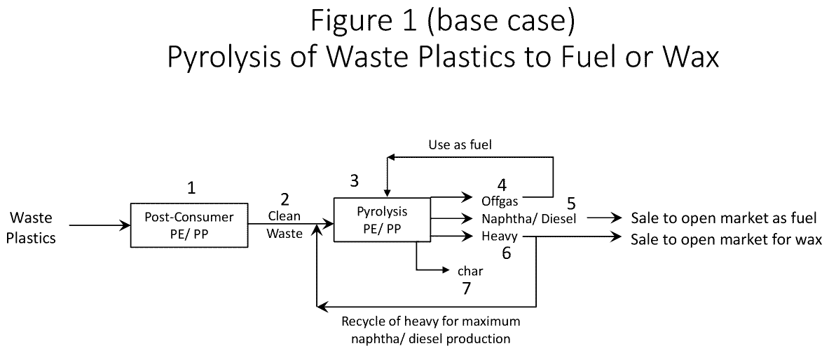

A simplified process diagram for the base case of a waste plastics pyrolysis process generally operated in the industry today is shown in FIG. 1. Generally, the waste plastics are sorted together 1. The cleaned plastic waste 2 is converted in a pyrolysis unit 3 to offgas 4 and pyrolysis oil (liquid product). The offgas 4 from the pyrolysis unit 3 is used as fuel to operate the pyrolysis unit. An on-site distillation unit separates the pyrolysis oil to produce naphtha and diesel 5 products which are sold to fuel markets. The heavy pyrolysis oil fraction 6 is recycled back to the pyrolysis unit 3 to maximize the fuel yield. Char 7 is removed from the pyrolysis unit 3. The heavy fraction 6 is rich in long chain, linear hydrocarbons, and is very waxy (i.e., forms paraffinic wax upon cooling to ambient temperature). Wax can be separated from the heavy fraction 6 and sold to the wax markets.

Using the present waste plastic/bio feed blend offers many advantages over the pyrolysis process. The present process does not pyrolyze the waste plastic. Rather, a blend of a bio feedstock and waste plastic is directly converted in a conversion unit.

The blend can be prepared in a hot blend preparation unit where the operating temperature is above the melting point of the plastic (about 120-300° C.), to make a hot, homogeneous liquid blend of plastic and bio oil. The hot homogeneous liquid blend of plastic and bio feedstock can be fed directly to the conversion units.

The preferred range of the plastic in the composition blend is about 1-20 wt. %. In one embodiment, the conditions for preparing the hot liquid blend include heating the blend above the melting point of the plastic while vigorously mixing with a bio feedstock. The process conditions can include heating to 250-550° F., a residence time of 5-240 minutes at the final heating temperature, and 0-10 psig atmospheric pressure. This can be done in the open atmosphere as well as preferably under an oxygen-free atmosphere.

Alternatively, a blend is prepared in a stable blend preparation unit where the hot homogeneous liquid blend is cooled to ambient temperature in a controlled manner to allow for easy storage and transportation. By using this method, a stable blend can be prepared at a remote facility away from a refinery and can be transported to the refinery unit. Then the stable blend is heated above the melting point of the plastic to feed to the refinery conversion unit. The stable blend is a physical mixture of micron size plastic particles finely suspended in the petroleum-based oil. The mixture is stable, and the plastic particles do not settle or agglomerate upon storage for extended period.

The present stable blend is made by a two-step process. The first step produces a hot, homogeneous liquid blend of plastic melt and bio feedstock. The preferred range of the plastic composition in the blend is about 1-20 wt %. In one embodiment, the conditions for preparing the hot liquid blend include heating the plastic above the melting point of the plastic while vigorously mixing with a bio feedstock. The preferred process conditions include heating to 250-550° F., a residence time of 5-240 minutes at the final heating temperature, and 0-10 psig atmospheric pressure. This can be done in the open atmosphere as well as preferably under an oxygen-free inert atmosphere.

In the second step, the hot blend is cooled down below the melting point of the plastic while continuously, vigorously mixing with the bio feedstock, and then further cooling to a lower temperature, preferably an ambient temperature, to produce a stable blend.

The resulting composition comprises a stable blend of a waste plastic and a bio feedstock for direct conversion of waste plastic in a conversion unit, such as a refinery process unit. The resulting composition is novel and offers many benefits.

The stable blend is made of bio feedstock and 1-20 wt % of plastic waste, wherein the plastic is mostly polyethylene, polypropylene and/or polystyrene, and the plastic is in the form of finely dispersed micron sized particles.

What is meant by heating the blend to a temperature above the melting point of the plastic is clear when a single plastic is used. However, if the waste plastic comprises more than one waste plastic, then the melting point of the plastic with the highest melting point is exceeded. Thus, the melting points of all plastics must be exceeded. Similarly, if the blend is cooled below the melting point of the plastic, the temperature must be cooled below the melting points of all plastics comprising the blend.

There are several advantages over pyrolysis realized in implementing these concepts.

The present process does not pyrolyze the waste plastic. Rather, a blend of a bio feedstock and waste plastic is prepared. Thus, the pyrolysis step can be avoided, which is a significant energy savings.

The stable blend of plastic and bio feedstock can be stored at ambient temperature and pressure for extended time periods. During the storage, no agglomeration of polymer and no chemical/physical degradation of the blend is observed. This allows easier handling of the waste plastic material for storage or transportation.

The stable blend can be handled easily by using standard pumps typically used in refineries or warehouses, or by using pumps equipped with transportation tanks. Depending on the blend, some heating of the blend above its pour point is required to pump the blend for transfer or for feeding to a conversion unit in a refinery. During the heating, no agglomeration of polymer is observed.

For feeding to a conversion unit, the stable blend is further heated above the melting point of the plastic to produce a homogeneous liquid blend of bio feedstock and plastic. The hot homogeneous liquid blend is fed directly to the oil refinery process units for conversion of waste plastics and bio feedstock to high-value, sustainable products with good yields.

Also compared with the pyrolysis unit, these blend preparation units operate at a much lower temperature (˜500-600° C. vs. 120-300° C.). Thus, the present process is a far more energy efficient process in preparing a refinery feedstock derived from waste plastic than a thermal cracking process such as pyrolysis.

The use of the present waste plastic/bio feedstock blend further increases the overall hydrocarbon yield obtained from the waste plastic. This increase in yield is significant. The hydrocarbon yield using the present blend offers a hydrocarbon yield that can be as much as 98%. To the contrary, pyrolysis produces a significant amount of light product from the plastic waste, about 10-30 wt. %, and about 5-10 wt. % of char. These light hydrocarbons are used as fuel to operate the pyrolysis plant, as mentioned above. Thus, the liquid hydrocarbon yield from the pyrolysis plant is at most 70-80%.

Also, when the present blend is passed into the refinery units, such as a FCC unit, only a minor amount of offgas is produced. Refinery units use catalytic cracking processes that are different from the thermal cracking process used in pyrolysis. With catalytic processes, the production of undesirable light-end byproducts such as methane and ethane is minimized. Refinery units have efficient product fractionation and are able to utilize all hydrocarbon products streams efficiently to produce high value materials. Refinery co-feeding will produce only about 2% of offgas (H₂, methane, ethane, ethylene). The C₃ and C₄ streams are captured to produce useful products such as circular polymer and/or quality fuel products. Thus, the use of the present petroleum/plastic blend offers increased hydrocarbons from the plastic waste, as well as a more energy efficient recycling process compared to a thermal process such as pyrolysis.

The present process converts single use waste plastic in large quantities by integrating the waste plastic blended with petroleum product streams into an oil refinery operation. The resulting processes produce the feedstocks for the polymers (liquid petroleum gas (LPG), C₃ olefin stream for a propylene polymerization unit), high quality gasoline, diesel and jet fuel, and/or quality base oil.

Generally, the present process provides a circular economy for polypropylene plants. Polypropylene is produced via polymerization of pure propylene. Clean propylene can be made from a propane dehydrogenation unit. Also, propylene can be obtained from an oil refinery fluid catalytic cracking (FCC) unit, which produces a mix of propylene and propane liquefied petroleum gas (LGP). Pure propylene is separated from the mix using a propane/propylene splitter, a high efficiency distillation column (PP splitter).

By adding refinery operations to upgrade the waste plastic to higher value products (gasoline, jet fuel and diesel, base oil) and to produce clean propylene for polypropylene polymer production, positive economics are realized for the overall process of recycled plastics to polypropylene products with product quality identical to that of the virgin polymer. And, by integrating the present recycle process with an oil refinery operation, a more energy efficient and effective process is achieved while avoiding any issues with the refinery operation.

The integration of a refinery operation becomes quite important in another aspect. Waste plastics contain contaminants, such as calcium, magnesium, chlorides, nitrogen, sulfur, dienes, and heavy components, which products cannot be used in a large quantity for blending in transportation fuels. It has been discovered that by having these products go through the refinery units, the contaminants can be captured in pre-treating units and their negative impacts diminished. The fuel components can be further upgraded with appropriate refinery units using chemical conversion processes, with the final transportation fuels produced in the integrated process being of higher quality and meeting the fuels quality requirements. The integrated process will generate a much cleaner and more pure propane stream for the propane dehydrogenation unit and ultimately for polypropylene production. These large on-spec productions allow “cyclical economy” for the recycle plastics to be feasible.

The carbon in and out of the refinery operations are “transparent,” meaning that all the molecules from the waste plastic do not necessarily end up in the exact olefin product cycled back to the polyolefin plants, but are nevertheless assumed as “credit” as the net “green” carbon in and out of the refinery is positive. With these integrated processes, the amount of virgin feeds needed for polypropylene plants are reduced significantly.

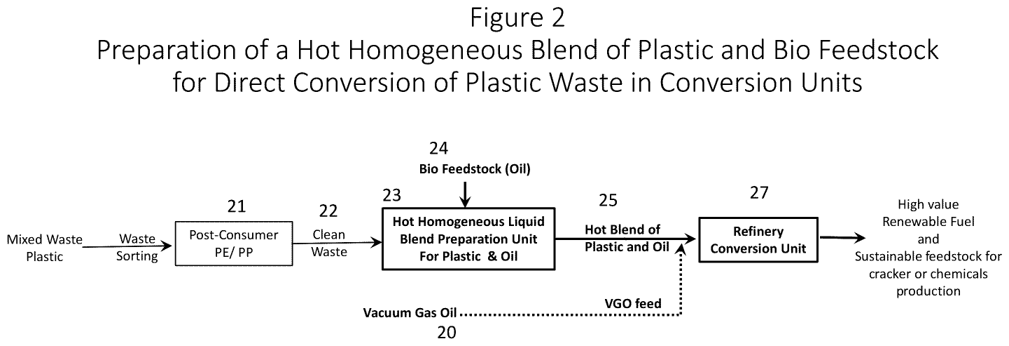

FIG. 2 illustrates a method for preparing a hot homogeneous blend of plastic and bio feedstock in accordance with the present process. The hot liquid blend can be used for direct injection to a conversion unit. The preferred range of the plastic composition in the blend is about 1-20 wt. %. If high molecular weight polypropylene (average molecular weight of 250,000 or greater) waste plastic or high-density polyethylene (density above 0.93 g/cc) is used as the predominant waste plastic, e.g., at least 50 wt. %, then the amount of waste plastic used in the blend is more preferably about 10 wt. %. The reason being that the pour point and viscosity of the blend would be high.

The preferred conditions for the blend preparation include heating the plastic above the melting point of the plastic while vigorously mixing with a bio feedstock. The preferred process conditions include heating to a 250-550° F. temperature, with a residence time of 5-240 minutes at the final heating temperature, and 0-10 psig atmospheric pressure. This can be done in the open atmosphere as well as preferably under an oxygen-free inert atmosphere.

Referring to FIG. 2 of the Drawings, a stepwise preparation process of preparing the blend of plastic and bio feedstock is shown. Mixed waste plastic is sorted to create post-consumer waste plastic 21 comprising polyethylene and/or polypropylene. The waste plastic is cleaned 22 and then mixed with a bio feedstock oil 24 in a hot blend preparation unit 23. After the mixing in 23, the hot homogeneous blend of the plastic and bio oil is recovered 25. Optionally a filtration device may be added (not shown) to remove any undissolved plastic particles or any solid impurities present in the liquid blend. The blend of the plastic and bio oil can then be passed to a catalytic conversion unit 27. In the present process, in one embodiment, the conversion unit is a refinery unit such as a FCC unit. Optionally, the conversion unit may co-process vacuum gas oil 20 or another refinery conventional feedstock.

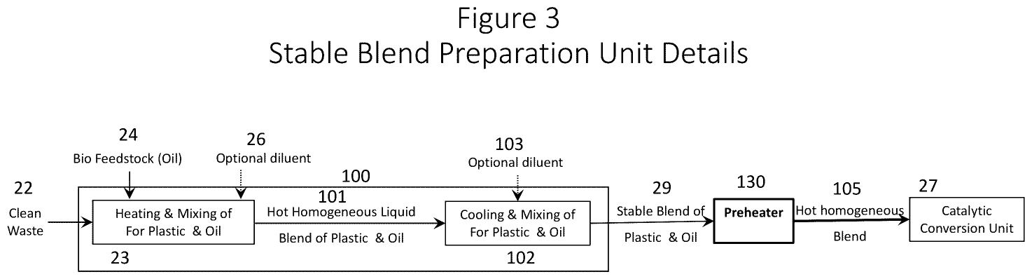

FIG. 3 illustrates a method for preparing a stable blend of plastic and oil for use in the present process. The stable blend is made in a stable blend preparation unit by a two-step process. The first step produces a hot, homogeneous liquid blend of plastic melt and bio feedstock, the step identical to the hot blend preparation described in FIG. 2. The preferred range of the plastic composition in the blend is about 1-20 wt. %. If high molecular weight polypropylene (average molecular weight of 250,000 or greater) waste plastic or high-density polyethylene (density above 0.93 g/cc) is used as the predominant waste plastic, e.g., at least 50 wt. %, then the amount of waste plastic used in the blend is more preferably about 10 wt. %. The reason being that the pour point and viscosity of the blend would be high.

The preferred conditions for the hot homogeneous liquid blend preparation include heating the plastic above the melting point of the plastic while vigorously mixing with a bio feedstock. The preferred process conditions include heating to a 250-550° F. temperature, with a residence time of 5-240 minutes at the final heating temperature, and 0-10 psig atmospheric pressure. This can be done in the open atmosphere as well as preferably under an oxygen-free inert atmosphere.

In the second step, the hot blend is cooled down below the melting point of the plastic while continuously vigorously mixing. An optional diluent can be added during the mixing. The further cooling is to a lower temperature, preferably ambient temperature, to produce a stable blend of plastic and oil.

It has been found that the stable blend is an intimate physical mixture of plastic and bio feedstock. The plastic is in a “de-agglomerated” state. The plastic maintains a finely dispersed state of solid particles in the bio feedstock at temperatures below the melting point of the plastic, and particularly at ambient temperatures. The blend is stable and allows easy storage and transportation. At a refinery, the stable blend can be heated in a preheater above the melting point of the plastic to produce a hot, homogenous liquid blend of the plastic and bio feedstock. The hot liquid blend can then be fed to a refinery unit, either alone or as a cofeed with conventional refinery feed.

In FIG. 3, further details of the stable blend preparation are shown. The stable blend is made in a stable blend preparation unit 100 by a two-step process. As shown, clean waste 22 is passed to the hot blend preparation unit 23. The selected plastic waste 22 is mixed with a bio feedstock oil 24 and heated above the melting point of the plastic in unit 23. The mixing is often quite vigorous. An optional diluent 26 can be added during the mixing. The mixing and heating conditions can generally comprise heating at a temperature in the range of about 250-550° F., with a residence time of 5-240 minutes at the final heating temperature. The heating and mixing can be done in the open atmosphere or under an oxygen-free inert atmosphere. The result is a hot, homogenous liquid blend of plastic and oil 101. Optionally a filtration device may be added (not shown) to remove any undissolved plastic particles or any solid impurities present in the hot homogeneous liquid blend.

The hot blend 101 is then cooled below the melting point of the plastic while continuing the mixing of the plastic and bio oil blend at unit 102. An optional diluent 103 can be added during the mixing and cooling. Cooling generally continues, usually to an ambient temperature, to produce a stable blend of the plastic and oil 29. At a refinery, the stable blend can be fed to a preheater 130, which heats the blend above the melting point of the plastic to produce a hot homogeneous mixture of plastic/oil blend 105, which is then fed to a refinery conversion unit 27. Optionally, the conversion unit may co-process vacuum gas oil or another conventional refinery feedstock.

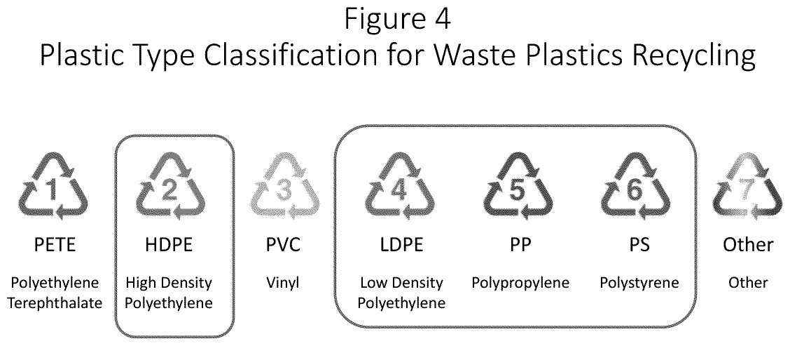

The preferred plastic starting material for the present process is sorted waste plastics containing predominantly polyethylene and polypropylene (plastics recycle classification types 2, 4, and 5). The pre-sorted waste plastics are washed and shredded or pelleted to feed to a blend preparation unit. FIG. 4 depicts the plastic type classification for waste plastics recycling. Classification types 2, 4, and 5 are high density polyethylene, low density polyethylene and polypropylene, respectively. Any combination of the polyethylene and polypropylene waste plastics can be used. For the present process, at least some polyethylene waste plastic is preferred. Polystyrene, classification 6, can also be present in a limited amount.

Proper sorting of waste plastics is very important in order to minimize contaminants such as N, Cl, and S. Plastics waste containing polyethylene terephthalate (plastics recycle classification type 1), polyvinyl chloride (plastics recycle classification type 3) and other polymers (plastics recycle classification type 7) need to be sorted out to less than 5%, preferably less than 1% and most preferably less than 0.1%. The present process can tolerate a moderate amount of polystyrene (plastics recycle classification type 6). Waste polystyrene needs to be sorted out to less than 20%, preferably less than 10% and most preferably less than 5%.

Washing of waste plastics can remove metal contaminants such as sodium, calcium, magnesium, aluminum, and non-metal contaminants coming from other waste sources. Non-metal contaminants include contaminants coming from the Periodic Table Group IV, such as silica, contaminants from Group V, such as phosphorus and nitrogen compounds, contaminants from Group VI, such as sulfur and oxygen compounds, and halide contaminants from Group VII, such as fluoride, chloride, and iodide. The residual metals, non-metal contaminants, and halides need to be removed to less than 50 ppm, preferentially less than 30 ppm and most preferentially to less than 5 ppm.

The term “bio” refers to biochemical and/or natural chemicals found in nature. Thus, a bio feedstock or bio oil would comprise such natural chemicals. The preferred starting bio feedstocks for the blend preparation include triglycerides and fatty acids, plant-derived oils such as palm oil, canola oil, corn oil, and soybean oil, as well as animal-derived fats and oils such as tallow, lard, schmaltz (e.g., chicken fat) and fish oil, and mixtures of these. In one embodiment, the bio feedstocks can comprise biomass pyrolysis oil prepared by pyrolyzing a bio feedstock material.

The most preferred bio feedstocks are palm oil and tallow, with a high degree of saturation exhibiting an iodine number of 91 or below (i.e., low degree of unsaturation). The iodine number (or iodine value) is a measure of the amount of unsaturation in fats, oils and waxes. It is determined by measuring the mass of iodine in grams consumed by 100 g of substance. A higher value means a substance is more unsaturated. It is similar to the use of the bromine number to measure unsaturation in petroleum samples.

It has been discovered that bio feedstocks with polyunsaturated fatty acids with a high iodine number, such as soybean oil (with 130 iodine number), do not make stable blends with plastic. However, a bio feedstock mixture consisting of low (≤70) and high (>70) iodine number bio feedstocks can make a stable blend with plastic. It has been discovered that bio feedstock mixtures with about a 95 iodine number or less make a stable blend with plastic. In one embodiment, the mixture of bio feedstocks exhibits an iodine number of 91 or less.

Further the plastic and bio feedstock blend can be blended with other diluent hydrocarbons, such as heptane, as needed to alter the properties of the blend, e.g. the viscosity or pour point, for easier handling or for processing. Preferred blending hydrocarbon feedstocks include standard petroleum-based feedstocks such as vacuum gas oil (VGO), an aromatic solvent or light cycle oil (LCO). In one embodiment, the blending hydrocarbon feedstock comprises atmospheric gas oil, VGO, or heavy stocks recovered from other refinery operations. In another embodiment, the blending hydrocarbon feedstock comprises LCO, heavy cycle oil (HCO), FCC naphtha, gasoline, diesel, toluene, or aromatic solvent derived from petroleum. A portion of the liquid FCC product (naphtha and LCO for example) could also be recycled to the blend in order to lower the viscosity. In one embodiment, no petroleum feedstocks are used, and only bio feedstocks are used in creating the blend and mixing with the blend.

While not wishing to be bound by a theory, the prepared stable blend is an intimate physical mixture of plastic and bio feedstock for catalytic conversion units. The present process produces a stable blend of bio feedstock and plastic wherein plastic is in a “de-agglomerated” state. This blend is stable and allows easy storage and transportation. At a refinery, the stable blend is preheated above the melting point of the plastic to produce a hot homogeneous liquid blend of plastic and bio feedstock, and then the hot liquid blend is fed to a conversion unit. Then both the bio feed and plastic are simultaneously converted in the conversion unit with typical refinery catalysts containing zeolite(s) and other active components such as silica-alumina, alumina and clay.

Catalytic conversion units such as a fluid catalytic cracking (FCC) unit, hydrocracking unit, and hydrotreating unit, convert the hot homogeneous liquid blend of plastic and bio feedstock in the presence of catalysts for simultaneous conversion of the plastic and bio feedstock. The presence of catalysts in the conversion unit allows conversion of the waste plastics to higher value products at a lower operating temperature than the typical pyrolysis temperature. For the hydroprocessing units (hydrocracking and hydrotreating units), hydrogen is added to units to improve the conversion of the plastics.

A fluid catalytic cracking process is the preferred mode of catalytic conversion of the stable blend. The catalyst selection is optimized to maximize monomer production for the manufacture of virgin plastics.

The yields of undesirable byproducts (offgas, tars, coke) are lower than the typical pyrolysis process. The blend may generate additional synergistic benefits coming from the interaction of plastic and bio feedstock during the conversion process.

The blend of plastic and bio feedstock allows more efficient recycling of waste plastics and enables truly circular and sustainable plastics and chemicals production. It is far more energy efficient than the current pyrolysis process and allows recycling with a lower carbon footprint. The improved processes allow the establishment of circular economy at a much larger scale by efficiently converting waste plastics back to the virgin quality polymers or value-added chemicals and fuels.

A dedicated conversion unit for conversion of plastic and bio feedstock blend will generate sustainable, low-carbon chemical intermediates and fuel without any petroleum feed stock usage.

Preferably, the plastic and bio feedstock blend can be fed to oil refinery conversion units for co-processing with petroleum-based oil. Refinery conversion units such as the fluid catalytic cracking (FCC) unit, hydrocracking unit, and hydrotreating unit are preferred for simultaneous conversion of the plastic, bio feedstock and petroleum-based oil.

In such a case, the refinery will generally have its own hydrocarbon feed flowing through the refinery units. For example, the hydrocarbon feed can be VGO. The flow volume of blend to the refinery units, such as an FCC unit, can comprise any practical or accommodating volume % of the total flow to the refinery units. Generally, the flow of the blend, for practical reasons, can be up to about 50 vol. % of the total flow, i.e., the refinery flow and the blend flow. In one embodiment, the flow of the blend is an amount up to about 100 vol. % of the total flow. The volume % of the blend will also depend on the ultimate end product desired. If aromatics and xylenes are the focal chemicals, then the blend flow % can be much higher, if not 100%. In another embodiment, the volume flow of the blend is an amount up to about 25 vol. % of the total flow. About 50 vol. % has been found to be an amount that is quite practical in its impact on the refinery while also providing excellent results and being an amount that can be accommodated. Avoiding any negative impact on the refinery and its products is important. If the amount of the plastic in the final blend (comprising the plastic/oil blend and co-feed petroleum) is greater than 20 wt. % of the final blend, difficulties in FCC unit operation might ensue. By the final blend is meant the present plastic/oil blend and any co-feed petroleum. The plastic/oil blend can comprise up to 100 vol. % of the feed to the refinery units.

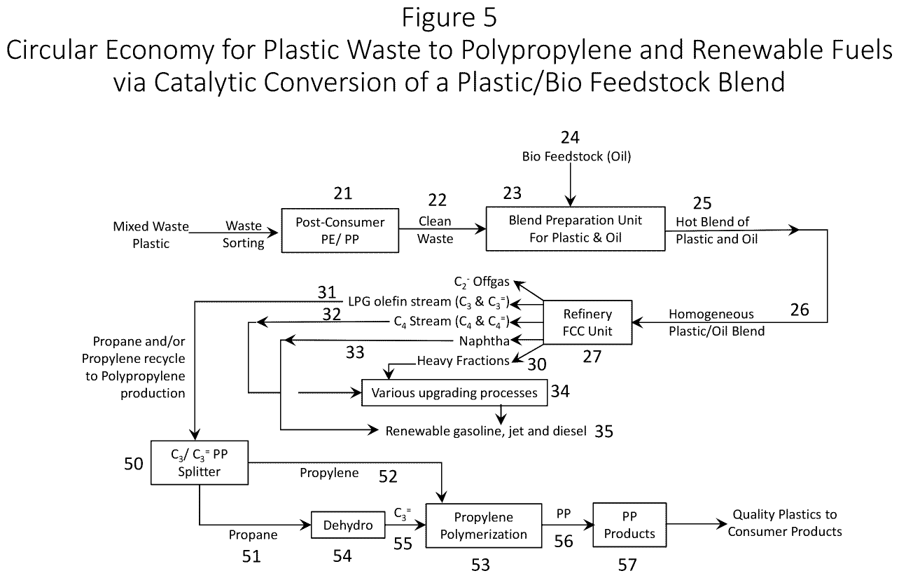

In FIG. 5, cracking of the plastic/bio oil hot blend 25, either alone or combined with a co-feed petroleum feed, can be passed via 26 to the conversion FCC unit 27. The numerals in FIG. 5, which are the same as in FIGS. 2 and 3, refer to the same streams or units. The FCC unit 27 produces liquefied petroleum gas (LPG) of C₃ and C₄ olefin/paraffin streams 31 and 32, and a naphtha 33 and heavy fraction 30. The C₃ olefin/paraffin mix stream of propane and propylene mix 31 can be sent to and separated by a propane/propylene splitter (PP splitter) 50 to produce pure steams of propane 51 and propylene 52. The propylene 52 is fed to propylene polymerization unit 52 to produce polypropylene.

The pure propane 51 may be fed to a propane dehydrogenation unit 54 to make additional propylene 55, and then ultimately polypropylene in the propylene polymerization unit 53.

Dehydrogenation of propane is practiced widely in the industry to produce propylene. The reaction is endothermic, conversion is maintained by multi-stage reactors and inter-stage heaters. The unit typically operates at high temperature (>900° F.) and low pressure (<50 psig) in the presence of noble metal (Pt) catalyst. The multi-stage process generates approximately 85% purity propylene/propane mixture. This stream is directed to a propane/propylene (PP) splitter which is a high efficiency distillation column. The splitter produces pure propylene stream with 99.5-99.8% purity.

The PP splitter unit and/or propane dehydrogenation unit can be located away from a refinery, near a refinery, or within a refinery. The propane/propylene mix is sent to the PP splitter by truck, barge, rail car or pipeline. It is preferred that the PP splitter unit and propane dehydrogenation unit are in close proximity to the refinery FCC unit.

The C₄ 32 and other hydrocarbon product streams, such as the heavy fraction 30 from the FCC unit 27, are sent to appropriate refinery units 34 for upgrading into clean gasoline, diesel, or jet fuel. The gasoline 33 from the FCC unit may be passed directly to a gasoline pool or further upgraded before sending to a gasoline pool (not shown in the figure).

The polypropylene polymer 56 made in the propylene polymerization unit 53, can be made into polypropylene products 57 that will then be further incorporated into consumer products.

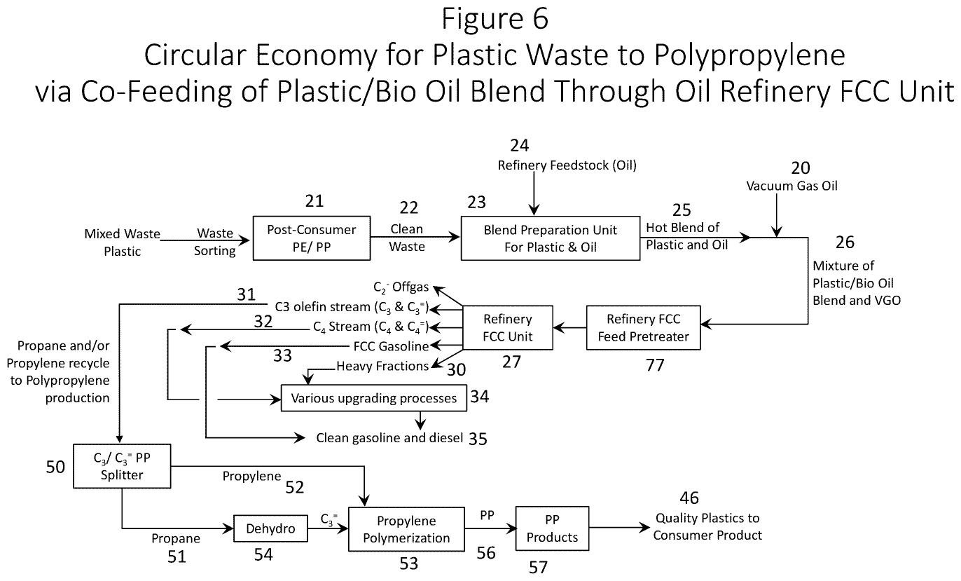

FIG. 6 shows a present integrated process such as that shown in FIG. 5, where a co-feed of a blend and hydrocarbon refinery flow 26 is sent first to a fluid catalytic cracking (FCC) feed pretreater unit 77. The numerals in FIG. 6 which are the same as in FIG. 5 refer to the same streams or refinery units.

The FCC Feed Pretreater typically uses a bimetallic (NiMo or CoMo) alumina catalyst in a fixed bed reactor to hydrogenate the feed with H₂ gas flow at a 660-780° F. reactor temperature and 1,000-2,000 psi pressure. The refinery FCC Feed Pretreater Unit is effective in removing sulfur, nitrogen, phosphorus, silica, dienes and metals that will hurt the FCC unit catalyst performance. Also, this unit hydrogenates aromatics and improves the liquid yield of the FCC unit.

The pretreated hydrocarbon from the feed pretreater unit 77 can be distilled to produce LPG, naphtha and heavy fraction. The heavy fraction is sent to FCC unit 27 for further production of C₃ 31, C₄ 32, FCC gasoline 33 and heavy fraction 30. A C₄ stream and naphtha from the feed pretreater unit can be passed to other upgrading processes within the refinery for production of gasoline, diesel and jet fuel. The C₃ 31 stream can be sent to propane/propylene splitter 50. The propylene 52 can be sent directly to propylene polymerization 53, whereas the propane 51 can be fed to a propane dehydrogenation unit 54 to make additional propylene for polymerization.

Polypropylene is produced via chain-growth polymerization from the monomer propylene. A Ziegler-Natta catalyst or metallocene catalyst is used to catalyze the polymerization of propylene to polypropylene polymer with desired properties. These catalysts are activated with special cocatalyst containing an organoaluminum compounds. The industrial polymerization processes uses either gas phase polymerization in a fluidized bed reactor or bulk polymerization in loop reactors. The gas phase polymerization typically runs at 50-90° C. temperature and a pressure of 8-35 atm pressure in the presence of H₂. The bulk polymerization proceeds at 60 to 80° C. and 30-40 atm pressure is applied to keep the propylene in liquid state.

The propylene polymerization unit 53 is preferably located near the refinery so that the feedstock (propylene) can be transferred via pipeline. For a petrochemical plant located away from the refinery, the feedstock can be delivered via truck, barge, rail car or pipeline.

The benefits of a circular economy and an effective and efficient recycling campaign are realized by the present integrated process.

The following examples are provided to further illustrate the present process and its benefits. The examples are meant to be illustrative and not limiting.

### Example 1: Properties of Plastic Samples, and Bio Feedstocks Used for Blend Preparations

Five plastic samples, low density polyethylene (LDPE, Plastic A), high density polyethylene (HDPE, Plastic B), two polypropylene samples with average molecular weight of ˜12,000 (PP, Plastic C) and ˜250,000 (PP, Plastic D), and polystyrene (PS, plastic E) were purchased, and their properties are summarized in Table 1.

TABLE 1
Properties of Plastics Used

| | LDPE | HDPE | PP | PP | PS |
|---|---|---|---|---|---|
| | (Plastic A) | (Plastic B) | (Plastic C) | (Plastic D) | (Plastic E) |
| Form | Pellets | Pellets | Pellets | Pellets | Pellets |
| Melt Index | 25 g/10 min | 12 g/10 min | — | 12 g/10 min | 2.0-4.0 g/10 min |
| | (190° C./2.16 kg) | (190° C./2.16 kg) | | (230° C./2.16 kg) | (200° C./5kg) |
| Melting Point, ° C. | 116 | 125-140 | 157 | 160-165 | 270 |
| Transition Temp, ° C. | 93, | 125, | 163, | — | 95 |
| | softening | softening | softening | | |
| Density, g/mL at 25° C. | 0.925 | 0.952 | 0.9 | 0.9 | 1.04 |
| Hardness | — | 66 | — | 100 | — |
| Average molecular | — | — | ~12,000 | ~250,000 | ~350,000 |
| weight, M_{w} | | | | | |
| Average molecular | — | — | ~5,000 | ~67,000 | ~170,000 |
| weight, Mₙ | | | | | |

Bio feedstocks used to prepare blends with plastic melts include palm oil, tallow and soybean oil, and their properties are shown in Table 2.

TABLE 2
Properties of Bio Feedstocks for Blend Preparation

| | Palm Oil, | Tallow, | Soybean Oil, |
|---|---|---|---|
| | Bio Feed #1 | Bio Feed #2 | Bio Feed #3 |
| Specific Gravity | 22.9 | 23.2 | 21.4 |
| Carbon, wt % | 76.8 | 76.65 | 78 |
| Hydrogen, wt % | 12.1 | 12 | 11.6 |
| Oxygen, wt % | 11.1 | 11.4 | 10.4 |
| H/C Molar Ratio | 1.87 | 1.86 | 1.77 |
| Iodine Number | 52 (measured) | 45 (measured) | 130 (measured) |
| | 49-55 (ref a) | 42-48 (ref b) | 120-139 (ref c) |
| Total S, ppm | <6 | 11 | <6 |
| Total N, ppm | 2.2 | 140 | 20.3 |
| Ni, ppm | <0.9 | <0.9 | <0.7 |
| V, ppm | <1.5 | <1 | <0.7 |
| Simdist, ° F. | | | |
| IBP (0.5%) | 959 | 679 | 996 |
| 5 wt % | 997 | 1062 | 1100 |
| 10 wt % | 1083 | 1083 | 1109 |
| 30 wt % | 1097 | 1099 | 1112 |
| 50 wt % | 1099 | 1111 | 1124 |
| 70 wt % | 1110 | 1113 | 1125 |
| 90 wt % | 1120 | 1125 | 1127 |
| 95 wt % | 1122 | 1134 | 1127 |
| FBP (99.5%) | 1137 | 1136 | 1149 |

a) Sanders T H (2003). “Ground Nut oil”. Encyclopedia of Food Sciences and Nutrition. Elsevier. pp. 2967-2974. doi: 10.1016/b0-12-227055-x/01353-5. ISBN 978-0-12-227055-0.
b) Andersen A J, Williams P N (4 Jul. 2016). Margarine. Elsevier. pp. 30-. ISBN 978-1-4831-6466-3.
c) O'Brien R D (5 Dec. 2008). Fats and Oils: Formulating and Processing for Applications (3 ed.). CRC Press. ISBN 978-1-4200-6167-3.

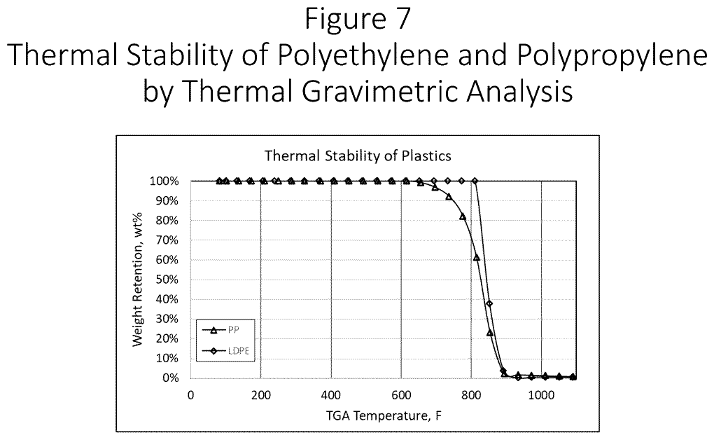

Thermal Gravimetric Analysis (TGA) was conducted with Plastic A (LDPE) and Plastic C (Polypropylene) to verify the plastic materials are thermally stable well above the melt preparation temperature. TGA results shown in FIG. 7 indicate the LPDE sample is stable up to 800° F. and the polypropylene sample up to 700° F.

### Example 2—Preparation of Stable Blends of Palm Oil and Plastic

Several blends of palm oil and the plastic were prepared by adding the plastic pellets (Plastic A through D) to a palm oil (Bio Feed #1).

The following procedure is used. At ambient temperature, the palm oil (waxy solids) was added to a beaker. The palm oil was heated with a heating mantle while stirring with a magnetic stirrer. The palm oil temperature was raised gradually to 270-400° F., and then pre-weighed plastic pellets (solids) were slowly added to the hot palm oil while stirring and heating. After the plastic pellets had dissolved, the stirred solution was then held at the final temperature for 30 additional minutes for blends with LDPE and Plastic C polypropylene, or for 60 minutes for blends with HDPE and Plastic D polypropylene. Then the blend was cooled down to ambient temperature while stirring. Visual observation indicated the blend is completely homogeneous. Upon cooling to ambient temperature, the blend of the plastic and palm oil showed the visual appearance of the waxy solid of the palm oil, but the hardening temperature (or solidification temperature) were different from the starting palm oil.

To assess material handling needs, a pour point (per ASTM D5950-14) and viscosity (per ASTM D445) of the blend were measured. In addition, the content of hot heptane insoluble material was measured per ASTM D3279 procedure. The hot heptane insoluble method determines the weight percent of material in oils that is insoluble in hot heptane at 80° C. The method isolates the insoluble material using 0.8-micron membrane filter. The heptane insoluble content provides information on non-dissolved plastic in the blend.

Material stability was observed by visual observation. The blend of plastic and palm oil was stable, and no change was observed for a 3-month period of observation.

Table 3 below summarizes the list of samples prepared and their properties.

TABLE 3
Preparation for Stable Blends of Plastic and Palm Oil

| | Wt % | Pour Point | Viscosity | Heptane | Storage |
|---|---|---|---|---|---|
| Example | Plastic in the Blend | ° C. | at 180° C. | Insoluble, wt % | Stability |
| Example 2-1 | None (base case) | 10 | 2.82 | 0.02 | Stable |
| Example 2-2 | 10 wt % LDPE (Plastic A) | 93 | 31.87 | 7.59 | Stable |
| Example 2-3 | 10 wt % HDPE (Plastic B) | — | — | — | Stable |
| Example 2-4 | 10 wt % PP (Plastic C) | 13 | 6.60 | 7.78 | Stable |
| Example 2-5 | 20 wt % PP (Plastic C) | — | — | — | Stable |
| Example 2-6 | 10 wt % PP (Plastic D) | — | — | — | Stable |

The pour point and viscosity values are used to guide equipment selection and operating procedure. The blends made with addition of plastic show moderate increases of pour point and viscosity compared with the pure bio base case. These changes can be handled with typical refinery operating equipment with minor or no modifications. The blend tank will be heated above the pour point to change the physical state of the blend into an easily transferable liquid. Then, the liquid blend can be transferred to a transportation vessel or to a refinery unit via pumping with a pump or via draining using gravity force or via transferring using a pressure differential.

The stable blends stay as physical mixtures at temperatures up to 80° C. The plastic was separated from the blend using the hot heptane insoluble test. At 80° C., all the wax in palm oil was dissolved in the heptane solvent (Example 2-1) and the heptane insoluble solids was only wt %. The weight percent of heptane insoluble material is coming from undissolved plastic that was filtered out with the 0.8-micron membrane filter. The amounts of recovered as solid material in Examples 2-2 and 2-4 showed 7.6 and 7.8 wt % heptane insoluble solids, similar to the amount of plastic added for the blend prep. The recovered amounts are less by 2.2-2.4 wt % suggesting there may be very fine particles in the blend that are sub-micron in size. The heptane insoluble results in Table 3 clearly indicated that the plastic is a physical mixture of solid particles dispersed in palm oil in the blend at 80° C. and that the bulk of plastic particles can be separated back with the 0.8-micron filter.

### Example 3—Preparation of Stable Blends of Tallow and Plastic

Several blends of tallow and plastic samples were prepared by adding the plastic pellets to a tallow feed (Bio Feed #2).

The procedure described in Example 2 was used for these blend preparations.

TABLE 4
Preparation for Stable Blends of Plastic and Tallow

| | Wt % | Pour Point | Viscosity | Heptane | Storage |
|---|---|---|---|---|---|
| Example | Plastic in the Blend | ° C. | at 180° C. | Insoluble, wt % | Stability |
| Example 3-1 | None (base case) | 30 | 2.96 | 0.04 | Stable |
| Example 3-2 | 10 wt % LDPE (Plastic A) | 93 | 33.01 | 7.67 | Stable |
| Example 3-3 | 10 wt % HDPE (Plastic B) | — | — | — | Stable |
| Example 3-4 | 10 wt % PP (Plastic C) | 29 | 7.65 | 8.64 | Stable |
| Example 3-5 | 20 wt % PP (Plastic C) | — | — | — | Stable |
| Example 3-6 | 10 wt % PP (Plastic D) | — | — | — | Stable |

The blends made with addition of plastic to tallow show moderate increases of pour point and viscosity compared with the pure bio base case, similar to the palm oil results shown in Example 2.

The weight percent of heptane insoluble material recovered as solid material matches well with the amounts of plastic added to the blend preparations. The base tallow (Example 3-1) has only 0.04 wt % the heptane insoluble while the stable blends (Example 3-2 and 3-4) showed 7.7 and 8.6 wt % heptane insoluble solid contents, similar to the amount of plastic added for the blend prep. The recovered amounts are less by 1.4-2.3 wt % suggesting there may be very fine particles in the blend that are sub-micron in size. The heptane insoluble results in Table 4 clearly indicate that the plastic is a physical mixture of solid particles dispersed in palm oil in the blend at 80° C., and that the bulk of plastic particles can be separated back with the 0.8-micron filter.

### Example 4—Preparation of Soybean Oil and Plastic Blends (Comparative Example)

An attempt was made to prepare blends of soybean oil (Bio Feed #3) and the plastic by using the procedures described in Example 2 with the plastic pellets (Plastic A through D). Surprisingly, soybean oil and plastic did not make hot homogeneous liquid melt upon heating together in the same manner as with palm oil or tallow. As the mixtures were heated above the melting point of the plastics the pellets did soften and lose their shape. But instead of forming an homogeneous liquid as in the other cases, the plastic melt formed a separate liquid phase from the soybean oil (SBO). Upon cooling, plastic phase was agglomerated and formed a large plastic solid. The likely explanation for this is that the SBO has a much higher degree of unsaturation, as is evident by its H/C ratio being lower than that of tallow or palm oil, and also from the measured iodine number. This also explains the reason most petroleum feeds, as well as highly saturated oils and fats as determined by the iodine number, readily dissolve plastics.

TABLE 5
Preparation for Stable Blends of Plastic and Soybean Oil

| | Wt % | |
|---|---|---|
| Example | Plastic in the Blend | Product description. |
| Example 4-1 | 10 wt % LDPE (Plastic A) | Stable blend cannot be made |
| Example 4-2 | 10 wt % HDPE (Plastic B) | Stable blend cannot be made |
| Example 4-3 | 10 wt % PP (Plastic C) | Stable blend cannot be made |
| Example 4-4 | 10 wt % PP (Plastic D) | Stable blend cannot be made |

### Example 5—Preparation of Soybean Oil, Palm Oil and Plastic Blends

A 1:1 weight mix of soybean oil and palm oil was prepared (mixed bio feedstock). With the mixed bio feedstock, successful blends of palm oil, soybean oil and the plastic were prepared by adding the plastic pellets (Plastic A and C) to the 1:1 mix of palm oil and soybean oil (Bio Feed #1 and Bio Feed #3). Therefore, SBO can be used as a component in the mixed bio feedstock. The stable blends showed good shelf life and did not show any changes for several months. These results show that soybean oil can also be used as a bio feedstock to prepare a stable blend with plastic, by lowering its unsaturation with another bio feedstock.

This test demonstrated an acceptable iodine number to make a successful stable blend of plastic and bio feedstock. The iodine number of 1:1 mixture of soybean oil and palm oil is estimated as 91. These results show that soybean oil or other highly unsaturated oils can also be used as a bio feedstock to prepare a stable blend with plastic, to the extent the overall unsaturation of mixed bio feedstock has an iodine number of 95 or less, and preferably 91 or less.

TABLE 6
Preparation for Stable Blends of Plastic, Soybean Oil and Palm Oil

| | Wt % | Pour Point | Viscosity | Heptane | Storage |
|---|---|---|---|---|---|
| Example | Plastic in the Blend | ° C. | at 180° C. | Insoluble, wt % | Stability |
| Example 5-1 | 10 wt % LDPE (Plastic A) | 17 | 31.98 | 6.78 | Stable |
| Example 5-2 | 10 wt % PP (Plastic C) | — | — | — | Stable |

### Example 6—Preparation of Soybean Oil, Tallow and Plastic Blends

A 1:1 mix of soybean oil and tallow was prepared. With the mixed bio feedstock, blends of tallow, soybean oil and the plastic were successfully prepared by adding the plastic pellets (Plastic A and C) to the 1:1 mix of tallow and soybean oil (Bio Feed #1 and Bio Feed #3). The stable blends showed good shelf life and no change was seen for several months. These results again show that soybean oil can also be used as a bio feedstock to prepare a stable blend with plastic, by lowering its unsaturation with another bio feedstock.

This test also shows an acceptable iodine number to make a successful stable blend of plastic and bio feedstock. The iodine number of 1:1 mixture of soybean oil and tallow is estimated as 88.

TABLE 7
Preparation for Stable Blends of Plastic, Soybean Oil and Tallow

| | Wt % | Stable Blend | Storage |
|---|---|---|---|
| Example | Plastic in the Blend | Preparation | Stability |
| Example 6-1 | 10 wt % LDPE (Plastic A) | Successful | Stable |
| Example 6-2 | 10 wt % PP (Plastic C) | Successful | Stable |

To study the impact of processing waste plastics and bio feedstocks in a FCC unit, laboratory tests of fluidized catalytic cracking (FCC) process were carried out with stable blends of plastic and bio feedstocks. Two FCC catalysts were used for the study: a ZSM-5 FCC catalyst made of ZSM-5 zeolite (10-membered ring medium pore zeolite) and a USY FCC catalyst made of USY (12-membered ring medium pore zeolite). Three bio feedstocks, palm oil, soybean oil and tallow were used.

The catalytic cracking experiments were carried out in an ACE (advanced cracking evaluation) Model C unit fabricated by Kayser Technology Inc. (Texas, USA). The reactor employed in the ACE unit was a fixed fluidized reactor with 1.6 cm ID. Nitrogen was used as fluidization gas and introduced from both bottom and top. The top fluidization gas was used to carry the feed injected from a calibrated syringe feed pump via a three-way valve. The experiments were carried out at atmospheric pressure and temperature of 975° F. For each experiment a constant amount of 1.5-gram of feed was injected at the rate of 1.2 gram/min for seconds. The cat/oil ratio was kept at 6. After 75 seconds of feed injection, the catalyst was stripped off by nitrogen for a period of 525 seconds. During the catalytic cracking and stripping process the liquid product was collected in a sample vial attached to a glass receiver, which was located at the end of the reactor exit and was maintained at −15° C. The gaseous products were collected in a closed stainless-steel vessel (12.6 L) prefilled with N2 at 1 atm. Gaseous products were mixed by an electrical agitator rotating at 60 rpm as soon as feed injection was completed. After stripping, the gas products were further mixed for 10 mins to ensure homogeneity. The final gas products were analyzed using a refinery gas analyzer (RGA). After the completion of stripping process, the in-situ catalyst regeneration was carried out in the presence of air at 1300° F. The regeneration flue gas passed through a catalytic converter packed with CuO pellets (LECO Inc.) to oxidize CO to CO₂. The flue gas was then analyzed by an online IR analyzer located downstream the catalytic converter. Coke deposited during cracking process was calculated from the CO₂ concentrations measured by the IR analyzer.

Gaseous products, mainly C₁ through C₇ hydrocarbons, were resolved in an RGA. The RGA is a customized Agilent 7890B GC equipped with three detectors, a flame ionization detector (FID) for hydrocarbons and two thermal conductivity detectors for nitrogen and hydrogen. A methanizer was also installed on the RGA to quantify trace amount of CO and CO₂ in the gas products when bio feedstocks, such as soybean oil, palm oil or tallow are cracked. Gas products were grouped into dry gas (C₂− hydrocarbons and hydrogen), LPG (C₃ and C₄ hydrocarbons). CO and CO₂ were excluded from dry gas. Their yields were reported separately. Liquid products were weighed and analyzed in a simulated distillation GC (Agilent 6890) using ASTM D2887 method. The liquid products were cut into gasoline (C₅− 430° F.), LCO (430-650° F.) and HCO (650° F.+). Gasoline (C₅+ hydrocarbons) in the gaseous products were combined with gasoline in the liquid products as total gasoline. Light ends in the liquid products (C₅−) were also subtracted from liquid products and added back to C₃ and C₄ species using some empirical distributions. Material balances were between 98% and 101% for most experiments.

Detailed hydrocarbon analysis (DHA) using Agilent 6890A and Hydrocarbon Expert software from Separation Systems Inc., FL were also performed on the gasoline portion of liquid products for PONA and octanes (RON and MON). DHA analysis on the gasoline portion in gaseous products were not performed. The DHA results, however, still provided valuable information to evaluate catalytic cracking product properties.

### Example 7—Direct Conversion of Plastic and Palm Oil Via FCC Using a ZSM-5 Catalyst

Laboratory tests of fluidized catalytic cracking (FCC) process were carried out with stable blends of plastic and bio feedstock (Examples 2-2 and 5-1) using an FCC catalyst made of ZSM-5 zeolite and the results are summarized in Table 8.

TABLE 8
Evaluation of Plastic and Palm Oil Cofeeding to FCC with ZSM-5 Catalyst

| Example No. | Example 7-1 | Example 7-2 | Example 7-3 |
|---|---|---|---|
| Feed | 100% Palm Oil | 10/90 wt % blend, | 10/45/45 wt % blend, |
| | (Example 2-1 Bio Oil) | LDPE/Palm Oil | LDPE/Palm Oil/SBO |
| | | (Example 2-2 Blend) | (Example 5-1 Blend) |
| Cat/Oil, wt/wt | 6.0 | 6.0 | 6.0 |
| Conversion, wt %* | 97.79 | 98.00 | 95.53 |
| Yields | | | |
| Coke, wt % | 1.45 | 1.67 | 2.04 |
| Total Dry Gas, wt % | 5.69 | 6.45 | 6.12 |
| Hydrogen | 0.05 | 0.06 | 0.06 |
| Methane | 0.20 | 0.29 | 0.23 |
| Ethane | 0.22 | 0.28 | 0.28 |
| Ethylene | 5.22 | 5.82 | 5.55 |
| Total LPG, wt % | 34.97 | 37.21 | 33.08 |
| Propane | 2.72 | 3.65 | 3.16 |
| Propylene | 16.28 | 16.49 | 14.98 |
| n-Butane | 0.93 | 1.27 | 1.08 |
| Isobutane | 1.36 | 1.82 | 1.50 |
| C4 olefins | 13.68 | 13.97 | 12.37 |
| Gasoline, wt % | 42.03 | 40.38 | 42.66 |
| Composition of Gasoline Fraction | | | |
| n-Paraffins, wt % | 2.20 | 1.40 | 1.35 |
| Iso-paraffins, wt % | 6.59 | 5.51 | 4.06 |
| Aromatics, wt % | 76.26 | 80.62 | 85.02 |
| Naphthenes, wt % | 1.90 | 2.27 | 1.86 |
| Olefins, wt % | 12.30 | 10.06 | 7.34 |
| Benzene, wt % | 7.67 | 7.39 | 8.24 |
| Toluene, wt % | 25.89 | 27.01 | 29.11 |
| Ethylbenzene, wt % | 5.99 | 6.19 | 5.96 |
| m-xylene, wt % | 5.45 | 6.49 | 7.65 |
| p-xylene, wt % | 17.43 | 17.76 | 15.30 |
| o-xylene, wt % | 1.99 | 2.33 | 2.13 |
| p-xylene/total xylenes | 70% | 67% | 61% |
| LCO, wt % (430-650 F.) | 1.67 | 1.44 | 2.62 |
| HCO, wt % (650 F.+) | 0.54 | 0.71 | 1.84 |
| Gasoline Octane Number** | 104.42 | 104.53 | 103.37 |
| Aromatics, wt % of feed | 32.05 | 32.56 | 36.27 |
| C3═/C3 | 86% | 82% | 83% |
| C4═/C4 | 86% | 82% | 83% |
| C4═/C3═ | 0.84 | 0.85 | 0.83 |

*Conversion—conversion of 430° F.⁺ fraction to 430° F.⁻
**Octane number, (R + M)/2, was estimated from detailed hydrocarbon GC of FCC gasoline.

The results in Table 8 show that blends of waste plastic with bio feedstocks (palm oil and soybean oil) convert well with a ZSM-5 containing FCC catalyst. Surprisingly, the medium-pore, 10-membered ZSM-5 catalyst can convert over 95 wt % of the plastic/bio feedstock blends at the typical FCC process conditions (Examples 7-1 through 7-3). The high conversions lead to very low yields of LCO and HCO. All these cases produced very high LPG and aromatics yields indicating this process can be used for production of feedstocks for polymer and chemical manufacturing without petroleum resources.

Addition of 10 wt % plastic to the palm oil caused only minor changes to the FCC unit performance in terms of the conversion and the yields of dry-gas and coke suggesting that coprocessing of waste plastic and bio feedstock is readily feasible (Example 7-1 vs. Example 7-2). While the 10 wt. % blending of low-density polyethylene (Plastic A) to palm oil led to only minor increases of coke and dry gas yields, significant positive increases in LPG yield (35 vs. 37 wt %) and total aromatics yield (76 vs. 81 wt % in the gasoline fraction) were observed. The significant increases in the LPG and aromatics yields from plastic containing blend was quite unexpected. This clearly indicates synergistic effects of the bio feedstock and plastic blend in providing increased yields of LPG and aromatics.

The 10 wt % plastic blending to the palm and soybean oil (Example 7-3) also showed consistent results of high LPG and aromatics production.

These results indicate the ZSM-5 catalyst made of medium pour size zeolite is a preferred catalyst for LPG olefin and aromatics production when converting a bio feed/plastic blend.

The high yield of LPG with this process is significant. The LPG and LPG olefins are desirable feedstocks for polyethylene and polypropylene production.

The high yield of aromatics with this process is also quite significant as these aromatics can be used of polystyrene or polyethylene terephthalate productions. Another surprising finding was the selectivity of the para-xylene relative to the total xylene. With the ZSM-5 catalyst, the xylene produced by this process is substantially para-xylene with about 61-70% para-xylene selectivity (relative to the total xylene production). Para-xylene is the most desirable xylene isomer for polyethylene terephthalate polymer manufacturing.

While this process is more suited for chemicals manufacturing, a portion of the products can be used to make premium gasoline fuel. The gasoline produced by this process has superior octane numbers of over 100 due to high aromatic contents in the gasoline fraction. The LCO and HCO yields are small due to high conversion.

### Example 8—Direct Conversion of Plastic and Palm Oil Via FCC Using a USY Catalyst

Laboratory tests of fluidized catalytic cracking (FCC) process were carried out with stable blends of plastic and palm oil (Examples 2-2 and 5-1) using an FCC catalyst made of USY zeolite and the results are summarized in Table 9.

TABLE 9
Evaluation of Plastic and Palm Cofeeding to FCC with USY Catalyst

| Example No. | Example 8-1 | Example 8-2 | Example 8-3 |
|---|---|---|---|
| Feed | 100% Palm Oil | 10/90 wt % blend, | 10/45/45 wt % blend, |
| | (Example 2-1 Oil) | LDPE/Palm Oil | LDPE/Palm Oil/SBO |
| | | (Example 2-2 Blend) | (Example 5-1 Blend) |
| Cat/Oil, wt/wt | 6.00 | 6.00 | 6.00 |
| Conversion, wt %* | 85.88 | 86.67 | 85.88 |
| Yields | | | |
| Coke, wt % | 6.39 | 6.74 | 7.01 |
| Total Dry Gas, wt % | 2.17 | 2.22 | 2.24 |
| Hydrogen | 0.04 | 0.03 | 0.04 |
| Methane | 0.68 | 0.74 | 0.72 |
| Ethane | 0.50 | 0.50 | 0.52 |
| Ethylene | 0.96 | 0.95 | 0.97 |
| Total LPG, wt % | 19.45 | 21.27 | 19.88 |
| Propane | 1.52 | 1.56 | 1.55 |
| Propylene | 6.31 | 6.76 | 6.27 |
| n-Butane | 1.08 | 1.18 | 1.16 |
| Isobutane | 4.39 | 4.98 | 4.56 |
| C4 olefins | 6.15 | 6.79 | 6.35 |
| Gasoline, wt % | 45.39 | 44.85 | 45.52 |
| Composition of Gasoline Fraction | | | |
| n-Paraffins, wt % | 3.83 | 3.29 | 2.96 |
| Iso-paraffins, wt % | 13.63 | 19.47 | 18.03 |
| Aromatics, wt % | 62.63 | 57.56 | 58.13 |
| Naphthenes, wt % | 3.85 | 4.65 | 6.46 |
| Olefins, wt % | 16.03 | 14.98 | 13.68 |
| Benzene, wt % | 1.36 | 1.28 | 1.49 |
| Toluene, wt % | 8.01 | 6.90 | 7.12 |
| Ethylbenzene, wt % | 3.12 | 2.68 | 2.84 |
| m-xylene, wt % | 7.55 | 6.53 | 6.26 |
| p-xylene, wt % | 2.82 | 2.16 | 2.24 |
| o-xylene, wt % | 3.62 | 3.13 | 2.96 |
| p-xylene/total xylenes | 20% | 19% | 20% |
| LCO, wt % (430-650 F.) | 10.89 | 10.03 | 11.84 |
| HCO, wt % (650 F.+) | 3.23 | 3.30 | 2.28 |
| Gasoline Octane Number** | 90.81 | 87.98 | 91.85 |
| Aromatics, wt % of feed | 28.43 | 28.17 | 26.46 |
| C3═/C3 | 81% | 81% | 80% |
| C4═/C4 | 53% | 53% | 53% |
| C4═/C3═ | 0.98 | 0.98 | 1.01 |

*Conversion—conversion of 430° F.⁺ fraction to 430° F.⁻
**Octane number, (R + M)/2, was estimated from detailed hydrocarbon GC of FCC gasoline.

The results in Table 9 show that blends of waste plastic with bio feedstocks (palm oil and soybean oil) convert well with a USY containing FCC catalyst. The overall conversions of the blends are 86-87% at the typical FCC process conditions (Examples 8-1 through 8-3). All these cases produced low dry-gas yield (undesirable product), and high LPG, gasoline and LCO yields (desirable product) indicating this process can be used for simultaneous production of feedstocks chemical manufacturing as well as premium renewable fuel without petroleum resources.

Addition of 10 wt % plastic to the palm oil caused only minor changes to the FCC unit performance in terms of the conversion and the yields of dry-gas and coke indicating that coprocessing of waste plastic and bio feedstock is readily feasible (Example 8-1 vs. 8-2). The 10 wt. % blending of low-density polyethylene (Plastic A) to palm oil had only minor effects on the product yields: LPG yield (19.5 vs. 21.3 wt %), gasoline yield (45.4 vs. 44.9 wt %), LCO (10.9 vs. 10.0 wt %) and HCO yields (3.2 vs. 3.3 wt %). However, a debit of gasoline octane number by 3 numbers (91 to 88) due to the paraffinic nature of the plastic was observed.

The 10 wt % plastic blending to the palm and soybean oil (Example 8-3) also showed consistent results of high LPG and gasoline production.

With the USY catalyst, a synergistic effect of bio feedstock with plastic was not observed, nor the para-xylene selectivity observed with a ZSM-5 catalyst shown in Example 7-2. Compared with the ZSM-5 catalyst (Example 8.2 vs. Example 7.2), the USY catalyst produced much higher yields of LCO (10.0 vs. 1.4 wt %) and HCO (3.3 vs. 0.7 wt %). In addition, USY showed much higher coke selectivity (6.7 vs 1.7 wt %) and lower offgas yield (2.2 vs. 6.5 wt %). These results indicate that the USY catalyst made of large pore size zeolite is a preferred FCC catalyst for simultaneous production of chemical feedstock and premium fuel.

A portion of the products can be used to make premium fuel. The gasoline produced by this process has octane numbers of 91 to 88. Due to paraffinic nature of the plastic, the addition of polyethylene plastic causes some decrease in octane number. With refinery blending flexibility, this octane number debit can be compensated with minor blending adjustments.

The high yield of LPG with this process is significant. The LPG and LPG olefins are desirable feedstock for polyethylene and polypropylene production.

The high yield of aromatics with this process is also significant as these aromatics can be used in polystyrene or polyethylene terephthalate productions.

### Example 9—Direct Conversion of Plastic and Tallow Via FCC Using ZSM-5 Catalysts

Laboratory tests of fluidized catalytic cracking (FCC) process were carried out with stable blends of plastic and bio feedstock (Examples 3-2, 3-4, and 6-1) using an FCC catalyst made of ZSM-5 zeolite and the results are summarized in Table 10.

TABLE 10
Evaluation of Plastic and Tallow Cofeeding to FCC with ZSM-5 Catalyst

| Example No. | Example 9-1 | Example 9-2 | Example 9-3 | Example 9-4 |
|---|---|---|---|---|
| Feed | 100% Tallow | 10/90 wt % blend, | 10/90 wt % blend, | 10/45/45 wt % blend, |
| | (Example 3-1) | LDPE/Tallow | PP/Tallow | LDPE/Tallow/SBO |
| | | (Example 3-2) | (Example 3-4) | (Example 6-1) |
| Cat/Oil, wt/wt | 6.0 | 6.0 | 6.0 | 6.0 |
| Conversion, wt %* | 97.87 | 98.04 | 96.67 | 95.12 |
| Yields | | | | |
| Coke, wt % | 0.87 | 1.99 | 1.01 | 1.40 |
| Total Dry Gas, wt % | 6.11 | 6.15 | 6.13 | 6.09 |
| Hydrogen | 0.06 | 0.06 | 0.06 | 0.06 |
| Methane | 0.25 | 0.27 | 0.30 | 0.27 |
| Ethane | 0.25 | 0.27 | 0.29 | 0.30 |
| Ethylene | 5.56 | 5.55 | 5.49 | 5.46 |
| Total LPG, wt % | 36.54 | 36.36 | 35.52 | 31.89 |
| Propane | 3.26 | 3.57 | 3.48 | 3.12 |
| Propylene | 16.45 | 15.96 | 15.60 | 14.21 |
| n-Butane | 1.09 | 1.25 | 1.19 | 1.05 |
| Isobutane | 1.56 | 1.77 | 1.71 | 1.39 |
| C4 olefins | 14.18 | 13.81 | 13.55 | 12.12 |
| Gasoline, wt % | 40.71 | 41.68 | 42.09 | 44.08 |
| Composition of Gasoline Fraction | | | | |
| n-Paraffins, wt % | 1.74 | 1.40 | 1.52 | 1.36 |
| Iso-paraffins, wt % | 4.68 | 5.52 | 5.32 | 4.38 |
| Aromatics, wt % | 81.47 | 80.57 | 80.32 | 85.24 |
| Naphthenes, wt % | 2.14 | 2.29 | 2.36 | 1.65 |
| Olefins, wt % | 9.38 | 10.10 | 10.05 | 6.78 |
| Benzene, wt % | 7.52 | 7.49 | 6.67 | 8.36 |
| Toluene, wt % | 27.99 | 27.02 | 25.61 | 29.86 |
| Ethylbenzene, wt % | 6.07 | 6.17 | 6.01 | 5.94 |
| m-xylene, wt % | 3.38 | 6.43 | 6.60 | 6.82 |
| p-xylene, wt % | 20.14 | 17.85 | 17.76 | 16.62 |
| o-xylene, wt % | 1.66 | 2.29 | 2.38 | 2.26 |
| p-xylene/total xylenes | 67% | 67% | 66% | 65% |
| LCO, wt % (430-650 F.) | 1.50 | 1.38 | 2.22 | 2.85 |
| HCO, wt % (650 F.+) | 0.63 | 0.58 | 1.11 | 2.03 |
| Octane Number** | 103.30 | 104.55 | 103.66 | 103.07 |
| Aromatics, wt % of feed | 32.80 | 33.58 | 33.81 | 37.58 |
| C3═/C3 | 83% | 82% | 82% | 82% |
| C4═/C4 | 84% | 82% | 82% | 83% |
| C4═/C3═ | 0.86 | 0.87 | 0.87 | 0.85 |

*Conversion—conversion of 430° F.⁺ fraction to 430° F.⁻
**Octane number, (R + M)/2, was estimated from detailed hydrocarbon GC of FCC gasoline.

The results in Table 10 show that blends of waste plastic with bio feedstocks (tallow and soybean oil) convert well with a ZSM-5 containing FCC catalyst. Similar to the co-processing with palm oil, ZSM-5 catalyst showed very high conversion of over 95 wt % of the plastic/bio feedstock blends at the typical FCC process conditions (Examples 9-1 through 9-4). The high conversions lead to very low yields of LCO and HCO. All these cases produced very high LPG and aromatics yields indicating that this process can be used for production of feedstocks for polymer and chemical manufacturing without petroleum resources.

Addition of 10 wt % polyethylene or polypropylene plastic to the tallow caused only minor changes to the FCC unit performance in terms of the conversion and the yields of dry-gas and coke suggesting that coprocessing of waste plastic and bio feedstock is readily feasible (Example 9-1 vs. 9-2 and 9-3). Unlike the palm oil case shown in Example 7-2, the 10 wt. % blending of low-density polyethylene (Plastic A) and polypropylene (Plastic C) to tallow did not show any synergistic effects in LPG and aromatics yields. The LPG yield (36.5 vs. wt %) and total aromatics yield (80.6 vs. 80.3-80.6 wt % in the gasoline fraction) were similar for the plastic co-feeding cases.

The 10 wt % plastic blending to the tallow and soybean oil (Example 9-4) also showed consistent results of high LPG and aromatics production, as well as high para-xylene selectivity.

The high yields of LPG and aromatics shown in Table 10 indicate again that the ZSM-5 catalyst made of medium pore size zeolite is a preferred catalyst for LPG olefin and aromatics production from a blend of plastic and bio feedstock such as tallow. With the ZSM-5 catalyst, the xylene produced by this process is substantially para-xylene with about 65-67% para-xylene selectivity (relative to the total xylene production).

### Example 10—Direct Conversion of Plastic and Tallow Via FCC Using USY Catalysts

Laboratory tests of fluidized catalytic cracking (FCC) process were carried out with stable blends of plastic and tallow bio feedstocks (Examples 3-2, 3-4, 6-1) using an FCC catalyst made of USY zeolite and the results are summarized in Table 11.

TABLE 11
Evaluation of Plastic and Tallow Cofeeding to FCC with USY Catalyst

| Example No. | Example 10-1 | Example 10-2 | Example 10-3 | Example 10-4 |
|---|---|---|---|---|
| Feed | 100% Tallow | 10/90 wt % blend, | 10/90 wt % blend, | 10/45/45 wt % blend, |
| | (Example 3-1) | LDPE/Tallow | PP/Tallow | LDPE/Tallow/SBO |
| | | (Example 3-2) | (Example 3-4) | (Example 6-1) |
| Cat/Oil, wt/wt | 6.0 | 6.0 | 6.0 | 6.0 |
| Conversion, wt %* | 85.99 | 86.51 | 85.86 | 85.43 |
| Yields | | | | |
| Coke, wt % | 6.18 | 6.98 | 6.19 | 6.79 |
| Total Dry Gas, wt % | 2.20 | 2.31 | 2.21 | 2.42 |
| Hydrogen | 0.03 | 0.03 | 0.03 | 0.04 |
| Methane | 0.70 | 0.79 | 0.73 | 0.78 |
| Ethane | 0.51 | 0.52 | 0.51 | 0.57 |
| Ethylene | 0.95 | 0.97 | 0.94 | 1.03 |
| Total LPG, wt % | 20.10 | 22.65 | 21.63 | 20.36 |
| Propane | 1.56 | 1.62 | 1.54 | 1.64 |
| Propylene | 6.44 | 7.16 | 6.80 | 6.41 |
| n-Butane | 1.13 | 1.23 | 1.14 | 1.22 |
| Isobutane | 4.73 | 5.34 | 5.12 | 4.62 |
| C4 olefins | 6.24 | 7.31 | 7.03 | 6.47 |
| Gasoline, wt % | 44.64 | 43.55 | 44.27 | 44.44 |
| Composition of Gasoline Fraction | | | | |
| n-Paraffins, wt % | 3.29 | 3.19 | 3.47 | 3.07 |
| Iso-paraffins, wt % | 19.47 | 19.07 | 18.97 | 19.60 |
| Aromatics, wt % | 57.56 | 58.02 | 54.96 | 59.78 |
| Naphthenes, wt % | 4.65 | 4.85 | 5.67 | 5.63 |
| Olefins, wt % | 14.98 | 14.63 | 16.49 | 11.03 |
| Benzene, wt % | 1.28 | 1.27 | 1.28 | 2.40 |
| Toluene, wt % | 6.90 | 7.05 | 6.88 | 8.89 |
| Ethylbenzene, wt % | 2.68 | 2.82 | 2.48 | 2.93 |
| m-xylene, wt % | 6.53 | 6.70 | 6.40 | 6.69 |
| p-xylene, wt % | 2.16 | 2.42 | 2.34 | 2.33 |
| o-xylene, wt % | 3.13 | 3.20 | 3.04 | 3.18 |
| p-xylene/total xylenes | 18% | 20% | 20% | 19% |
| LCO, wt % (430-650 F.) | 10.54 | 10.20 | 10.98 | 11.01 |
| HCO, wt % (650 F.+) | 3.47 | 3.29 | 3.16 | 3.56 |
| Octane Number** | 87.98 | 87.85 | 92.25 | 88.04 |
| Aromatics, wt % of feed | 25.69 | 25.27 | 24.33 | 26.57 |
| C3═/C3 | 81% | 82% | 82% | 80% |
| C4═/C4 | 52% | 53% | 53% | 53% |
| C4═/C3═ | 0.97 | 1.02 | 1.03 | 1.01 |

*Conversion—conversion of 430° F.⁺ fraction to 430° F.⁻
**Octane number, (R + M)/2, was estimated from detailed hydrocarbon GC of FCC gasoline.

The results in Table 11 show that blends of waste plastic with bio feedstocks (tallow and soybean oil) convert well with a USY containing FCC catalyst. The overall conversions of the blends are 85-87% at the typical FCC process conditions (Examples 10-1 through 10-4). All these cases produced low dry-gas yield (undesirable product), and high LPG, gasoline and LCO yields (desirable product) indicating that this process can be used for simultaneous production of feedstocks for chemical manufacturing as well as premium fuel without petroleum resources.

Addition of 10 wt % plastic to the tallow caused only minor changes to the FCC unit performance in terms of the conversion and the yields of dry-gas and coke indicating that coprocessing of waste plastic and bio feedstock is readily feasible (Example 10-1 vs. 10-2 and 10-3). The 10 wt. % blending of low-density polyethylene (Plastic A) or polypropylene (Plastic C) to tallow increased the LPG yield slightly (20.1 vs. 21.6-22.6 wt %) while having only minor effects on the other product yields: gasoline yield (44.6 vs. 43.6-44.3 wt %), LCO (10.5 vs. wt %) and HCO yields (3.5 vs. 3.2-3.3 wt %). No debit in gasoline octane number was observed with co-feeding of polyethylene and a 2 number gasoline octane number increase with co-feeding of polypropylene (88.0 vs. 87.9 vs. 92.3).

These results indicate that the USY catalyst made of large pore size zeolite is a preferred catalyst for simultaneous production of chemical feedstock and premium renewable fuels.

As used in this disclosure the word “comprises” or “comprising” is intended as an open-ended transition meaning the inclusion of the named elements, but not necessarily excluding other unnamed elements. The phrase “consists essentially of” or “consisting essentially of” is intended to mean the exclusion of other elements of any essential significance to the composition. The phrase “consisting of” or “consists of” is intended as a transition meaning the exclusion of all but the recited elements with the exception of only minor traces of impurities.

All patents and publications referenced herein are hereby incorporated by reference to the extent not inconsistent herewith. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise that as specifically described without actually departing from the spirit and scope of the present invention as defined by the appended claims.

What is claimed is:

1. A process for converting waste plastic into recycle for polypropylene polymerization comprising:
(a) selecting waste plastics comprising polyethylene and/or polypropylene;
(b) preparing a blend of a bio feedstock and the selected waste plastic with the blend comprising about 20 wt. % or less of the selected waste plastic;
(c) passing the blend at a temperature above the melting point of the plastic to a FCC unit;
(d) recovering a C₃ olefin/paraffin mixture from the FCC unit;
(e) separating the C₃ paraffin and C₃ olefin into different fractions; and
(f) passing the C₃ olefin to a propylene polymerization reactor.

2. The process of claim 1, wherein a gasoline and heavy fraction is recovered from the FCC unit.

3. The process of claim 1, wherein the blend of (b) is a hot homogeneous blend of waste plastic and bio oil.

4. The process of claim 1, wherein the blend of (b) is a stable blend of waste plastic and bio oil.

5. The process of claim 1, wherein polypropylene products are prepared from the polymerized propylene.

6. The process of claim 1, wherein the waste plastics selected in (a) comprise plastics from classification group 2, 4, and/or 5.

7. The process of claim 2, wherein the gasoline recovered from the FCC unit is sent to a gasoline blending pool.

8. The process of claim 1, wherein a C₄ stream and a heavy fraction are recovered from the FCC unit distillation column and further processed in the refinery to clean gasoline, diesel, or jet fuel.

9. The process of claim 1, wherein the volume flow of the blend to the FCC unit in (c) comprises up to 100 volume % of the total hydrocarbon flow to the FCC unit.

10. The process of claim 1, wherein the volume flow of the blend to the FCC unit in (c) comprises up to 50 volume % of the total hydrocarbon flow to the FCC unit.

11. The process of claim 10, wherein the blend flow comprises up to 25 volume % of the total flow to the FCC unit.

12. The process of claim 1, wherein the blend of bio feedstock and selected waste plastic in (b) is prepared by heating the waste plastic and bio feedstock mixture above the melting point of the plastic, and then cooling the blend to a temperature below the melting point of the waste plastic.

13. The process of claim 1, wherein the bio feedstock comprises triglycerides and/or fatty acids.

14. The process of claim 1, wherein the bio feedstock comprises plant-derived oils and/or animal-derived fats and oils.

15. The process of claim 14, wherein the plant derived oils comprise palm oil, canola oil, corn oil, soybean oil, or a mixture thereof.

16. The process of claim 14, wherein the bio feedstock comprises tallow, lard, schmaltz, fish oil, or a mixture thereof.

17. The process of claim 1, wherein the bio feedstock comprises palm oil, tallow, soybean oil or a mixture thereof.

18. The process of claim 1, wherein the bio feedstock comprise biomass pyrolysis oil.

19. The process of claim 1, wherein the bio feedstock comprises a bio feedstock or mixture of bio feedstocks having an iodine number of 95 or less.

20. The process of claim 19, wherein the iodine number is 91 or less.

21. The process of claim 19, wherein the bio feedstock comprises a mixture of a bio oil or fat exhibiting an iodine number of 70 or below, and a bio oil or fat exhibiting an iodine number above 70.

22. The process of claim 1, wherein a stream of petroleum feedstock comprising LCO or gasoline is added to reduce the blend viscosity.

23. The process of claim 1, wherein the blend passed to the FCC unit is mixed with a petroleum based feedstock

24. The process of claim 23, wherein the petroleum feedstock comprises from 1-50 vol % of the mixed blend.

25. The process of claim 24, wherein the petroleum based feedstock comprises atmospheric gas oil, vacuum gas oil (VGO), atmospheric residue, a petroleum derived oil, a petroleum based material, and/or heavy stock recovered from refinery operations.

26. The process of claim 24, wherein the petroleum based feedstock comprises light cycle oil (LCO), heavy cycle oil (HCO), FCC naphtha, gasoline, diesel, toluene, and/or aromatic solvent derived from petroleum.

27. The process of claim 1, wherein the blend passed to the FCC unit is mixed with a recycle stream to thereby lower the blend's viscosity.

28. Polypropylene products prepared by the process of claim 5.

29. Polypropylene prepared by the process of claim 1.
